(19) United States
(12) Patent Application Publication
(10) Pub. No.: US 2022/0176274 A1
(43) Pub. Date: Jun. 9, 2022

(54) METHOD OF SEPARATING HEMATOPOIETIC STEM CELLS FROM UMBILICAL CORD BLOOD

(71) Applicant: Hunan Yuanpin Cell Technology Co. Ltd, Changsha City (CN)

(72) Inventors: Cheng Wang, Changsha City (CN); Yaping Yang, Changsha City (CN); Yu Liao, Changsha City (CN); Lang Zhang, Changsha City (CN); Dan Wang, Changsha City (CN)

(21) Appl. No.: 17/351,150

(22) Filed: Jun. 17, 2021

(30) Foreign Application Priority Data
Dec. 9, 2020 (CN) 202011426548.4

(51) Int. Cl. B01D 17/02; B01D 17/12; C12N 5/0789

(52) U.S. Cl. CPC B01D 17/0217; B01D 17/12; C12N 5/0647; C12N 2523/00; C12N 2500/62; C12N 2500/34; B01D 2221/10

(57) ABSTRACT

The present disclosure provides a method of separating hematopoietic stem cells from umbilical cord blood, including the following steps: a hydroxyethyl starch solution is added into cord blood and mixed uniformly, then centrifuged to get an upper liquid layer and a lower erythrocyte layer; the liquid in the upper layer and the erythrocytes in the lower layer are centrifuged respectively; an upper plasma layer and a basal cell layer are obtained after the centrifugation of the upper liquid layer, the cells in the basal layer are resuspended; a superficial buffy-coat layer is obtained after the centrifugation of the erythrocytes, and the superficial buffy-coat layer is metered with the plasma in the upper layer, and then centrifuged to get a lower cell layer, which is precipitated to remove erythrocytes and then resuspended; the above resuspended cells are added into freezing medium, mixed uniformly and then stored in a liquid nitrogen tank.

### CROSS REFERENCE TO RELATED APPLICATION(S)

This patent application claims the benefit and priority of Chinese Patent Application No. 202011426548.4, filed on Dec. 9, 2020, the disclosure of which is incorporated by reference herein in its entirety as part of the present application.

### TECHNICAL FIELD

The present disclosure relates to the field of biotechnology, and particularly relates to a method of separating hematopoietic stem cells from umbilical cord blood.

### BACKGROUND ART

Cord blood, also called umbilical cord blood, refers to the blood remaining in the placenta and umbilical cord after the fetus is delivered and the umbilical cord is ligated and dissected, which is usually discarded. It has been found in the recent ten years of researches that, cord blood contains hematopoietic stem cells that can be used to rebuild human hematopoietic and immune systems, and used for the transplantation of hematopoietic stem cells and the treatment of various diseases. Therefore, cord blood has become an important source of hematopoietic stem cells, especially a source of unrelated-hematopoietic stem cells, and also a very important human biological resource.

Cord blood contains a large amount of stem cells, which are the seed of life, and can differentiate into various cells of the body and bear various different fruits including blood cells, nerve cells, bone cells and the like. With the development of science and technology, medical specialists have studied a method of treating diseases with stem cells in the umbilical cord blood. Stem cells are non-specialized cells which have the ability of self-renewal, high proliferation and multiple differentiation potentials. These cells can maintain the character and number of their own cells by division, and also can further differentiate into various tissue cells, thereby playing positive roles in tissue repair and other respects. It has been found in medical researches over the last three decades that, cord blood contains abundant hematopoietic stem cells (HSC), which can be used to rebuild human hematopoietic and immune systems, and used for the transplantation of hematopoietic stem cells and the treatment of diseases of blood system and immune system as well as inherited metabolic diseases and congenital diseases. Therefore, cord blood has become an important source of hematopoietic stem cells and has been widely applied in clinic, so it is a valuable human biological resource.

The methods for separating stem cells from umbilical cord blood at home and abroad commonly employ magnetic bead/flow cytometry cell sorting, cord blood banking instrument/hydroxyethyl starch precipitation and density gradient centrifugation. Due to different purposes, each method has its own characteristics. Magnetic bead or flow cytometry cell sorting utilizes corresponding monoclonal antibodies for separation to get a more pure cell population, but the yield of cells is low, so they are suitable for analysis and scientific research applications. Separation of stem cells from cord blood bank commonly uses processes of hydroxyethyl starch precipitation and instrument centrifugation, by which a large number of cells containing a variety of ingredients are separated, but there are a lot of erythrocytes mixed within them, so these processes are suitable for mass separation and storage of stem cells.

### SUMMARY

To overcome the defects in the prior art, the present disclosure is intended to provide a method of separating hematopoietic stem cells from umbilical cord blood so as to obtain cells in good condition and high vitality at a low separation cost and a high separation efficiency.

To achieve the above objective, the present disclosure employs the following technical schemes:

A method of separating hematopoietic stem cells from umbilical cord blood, including the following steps:

a) a hydroxyethyl starch solution of approximately 6% is added into cord blood and mixed uniformly, in which the volume ratio of the hydroxyethyl starch solution to the cord blood is approximately 4:1;

b) after mixing uniformly, they are divided into centrifuge tubes and let stand, then centrifuged to get an upper liquid layer and a lower erythrocyte layer;

c) the liquid in the upper layer and the erythrocytes in the lower layer are centrifuged respectively, an upper plasma layer and a basal cell layer are obtained after the centrifugation of the upper liquid layer, and the cells in the basal layer are resuspended with the plasma in the upper layer;

d) a superficial buffy-coat layer is obtained after the centrifugation of the erythrocytes, and the superficial buffy-coat layer is metered with the plasma in the upper layer, and then centrifuged to get a lower cell layer, which is precipitated to remove erythrocytes and then resuspended with the plasma in the upper layer;

e) the resuspended cells from steps (c) and (d) are combined together, and metered by adding the plasma in the upper layer, then a freezing medium is added and mixed uniformly before transferring into a freezing bag; and

f) cooling with a program-controlled cooling machine and then transferring into a liquid nitrogen tank for storage.

Illustratively, in the step (b), the volume of the centrifuge tubes is approximately 50 mL, the centrifugal force is approximately 50 g, and the time for centrifugation is approximately 10 min.

Illustratively, the operations of separation after centrifugation in the step (b) are as below: gently pipetting the liquid in the upper layer in the centrifuge tubes with an electric pipette gun equipped with a 10 ml pipette, and gently pipetting the last approximately 0.5 ml liquid in the upper layer with a 1 ml manual pipette, so as to pipette the buffy-coat layer out as much as possible and pipette the erythrocytes in the lower layer as little as possible.

Illustratively, in the step (c), the centrifugal force for the centrifugation of the upper liquid layer and the lower erythrocyte layer is approximately 747 g, and the time for centrifugation is approximately 10 min.

Illustratively, in the step (d), the superficial buffy-coat layer is metered with the plasma in the upper layer to approximately 30 mL, the centrifugal force is approximately 747 g, and the time for centrifugation is approximately 10 min.

Illustratively, in the step (e), the plasma in the upper layer is added for metering to approximately 20 mL.

Illustratively, the freezing medium used in the step (e) is prepared as below: dextran is mixed with dimethyl sulfoxide (DMSO) at a ratio of approximately 1:1, with dextran firstly added and then DMSO, and then precooled to get the freezing medium.

Illustratively, in the step (f), the temperature for storage in the liquid nitrogen tank is approximately −196° C.

Beneficial effects: The method of separating hematopoietic stem cells from cord blood of the present disclosure can separate hematopoietic stem cells from cord blood in a maximum proportion, the separation process of which is simple and easy to operate, the separated cells are highly active, the separation cost is low, the separation efficiency is high, and the recovery rate of cells can be thus improved. This method is simple and operable, with good stability and repeatability, so it can be widely applied.

### DETAILED DESCRIPTION OF THE EMBODIMENTS

The technical schemes of the present disclosure will be described clearly and completely below in combination with the following embodiment. Obviously, the described embodiment is only a part of embodiments of the present disclosure, but not all the embodiments.

### Embodiment 1

The present disclosure provides a method of separating hematopoietic stem cells from umbilical cord blood, including the following steps:

a) a hydroxyethyl starch solution of approximately 6% was added into cord blood and mixed uniformly for 5 min, in which the volume ratio of the hydroxyethyl starch solution to the cord blood was approximately 4:1;

b) after mixing uniformly, they were divided into 50 mL centrifuge tubes and let stand for 10 min, then centrifuged into two layers at a centrifugal force of approximately 50 g for approximately 10 min; the liquid in the upper layer in the centrifuge tubes was gently pipetted with an electric pipette gun equipped with a 10 ml pipette, and the last approximately 0.5 ml liquid in the upper layer was gently pipetted with a 1 ml manual pipette, so as to pipette the buffy-coat layer out as much as possible and pipette the erythrocytes in the lower layer as little as possible, thus obtaining the liquid in the upper layer and the erythrocytes in the lower layer;

c) the liquid in the upper layer and the erythrocytes in the lower layer were centrifuged at a centrifugal force of approximately 747 g for approximately 10 min, respectively; an upper plasma layer and a basal cell layer were obtained after the centrifugation of the upper liquid layer, the plasma in the upper layer was transferred into 50 ml centrifuge tubes as reserved samples and stored in a refrigerator at approximately −80° C. after labeling the serial number of the cell collection and the freezing date; and the cells in the basal layer were resuspended with approximately 2 mL plasma in the upper layer;

d) a superficial buffy-coat layer was obtained after the centrifugation of the erythrocytes, and the superficial buffy-coat layer was transferred into a new centrifuge tube and metered with the plasma in the upper layer to approximately 30 mL, and then centrifuged at a centrifugal force of approximately 747 g for approximately 10 min to get a lower cell layer, which was precipitated to remove erythrocytes and then resuspended with approximately 2 mL plasma in the upper layer;

e) the resuspended cells from steps (c) and (d) were combined together, and metered to 20 mL by adding the plasma in the upper layer, then approximately 5 mL precooled freezing medium was added and mixed uniformly before transferring into a freezing bag; the freezing medium was prepared as below: dextran was mixed with dimethyl sulfoxide (DMSO) at a ratio of approximately 1:1, with dextran firstly added and then DMSO, and then precooled to get the freezing medium;

f) opening the outer package of the syringe, the cell suspension added with the freezing medium was drawn with the syringe and injected slowly into a freezing bag; air in the freezing bag was expelled to prevent the bag from bursting when thawing; the pipe of the freezing bag was heat sealed at the end near the bag and at the T-connector; the upper 10 cm pipe formed by heat sealing was cut off and put into a freezing box together with the freezing bag, which was used for counting and determining the viability of cells during thawing; and

the serial numbers of the umbilical cord blood collection were written on the freezing bags with a marking pen, and the same serial numbers were marked on the freezing boxes with a marking machine at the same time; the freezing boxes charged with the umbilical cord blood were placed into the freezing case of a program-controlled cooling machine, and separated and frozen according to the procedures in PS-SMP-CP005 Standard Handling Procedures for Cell Separation and Freezing; the freezing boxes were taken out and placed in the freezing frames that had been written the serial numbers (Tank No., Region No. and Frame No.) with a paint marker, and then placed into a liquid nitrogen transfer tank at approximately −196° C. for temporary storage.

The frozen cells were thawed, and the number and recovery rate of the cells were calculated, with the results as shown in Table 1.

TABLE 1
Recovery rate of separating hematopoietic stem
cells from umbilical cord blood

| | | | Number of | |
|---|---|---|---|---|
| | | Initial | hematopoietic | |
| | | total | stem cells | |
| | Whole | number | after | Recovery |
| | blood | of cells | separation | rate |
| Embodiment 1 | 7.80 | 4.13 | 4.00 | 96.94% |

As can be seen from the above table, by using the method of separating hematopoietic stem cells from umbilical cord blood of the present disclosure, hematopoietic stem cells can be efficiently separated from whole blood, plasma can be removed as much as possible by hydroxyethyl starch, so as to improve the separation efficiency and reduce the loss rate of cells, thus reducing the freezing volume and decreasing the cost; in addition, the recovery rate of hematopoietic stem cells obtained after the final separation can reach approximately 96.94%.

The foregoing is only preferable implementation of the present disclosure, but the protection scope of the present disclosure is not limited to this. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances (e.g. 1-2%) of the system. Equivalent replacements or variations can be made by any persons familiar with skills in the art within the technical range disclosed in the present disclosure according to the technical schemes and inventive concepts of the present disclosure, which should be all covered within the protection scope of the present disclosure.

What is claimed is:

1. A method of separating hematopoietic stem cells from umbilical cord blood, wherein, comprising the following steps:
a) a hydroxyethyl starch solution of approximately 6% is added into cord blood and mixed uniformly, in which the volume ratio of the hydroxyethyl starch solution to the cord blood is approximately 4:1;
b) after mixing uniformly, they are divided into centrifuge tubes and let stand, then centrifuged to get an upper liquid layer and a lower erythrocyte layer;
c) the liquid in the upper layer and the erythrocytes in the lower layer are centrifuged respectively, an upper plasma layer and a basal cell layer are obtained after the centrifugation of the upper liquid layer, and the cells in the basal layer are resuspended with the plasma in the upper layer;
d) a superficial buffy-coat layer is obtained after the centrifugation of the erythrocytes, and the superficial buffy-coat layer is metered with the plasma in the upper layer, and then centrifuged to get a lower cell layer, which is precipitated to remove erythrocytes and then resuspended with the plasma in the upper layer;
e) the resuspended cells from steps (c) and (d) are combined together, and metered by adding the plasma in the upper layer, then a freezing medium is added and mixed uniformly before transferring into a freezing bag; and
f) cooling with a program-controlled cooling machine and then transferring into a liquid nitrogen tank for storage.

2. The method of separating hematopoietic stem cells from umbilical cord blood according to claim 1, wherein, in the step (b), the volume of the centrifuge tubes is approximately 50 mL, the centrifugal force is approximately 50 g, and the time for centrifugation is approximately 10 min.

3. The method of separating hematopoietic stem cells from umbilical cord blood according to claim 1, wherein, the operations of separation after centrifugation in the step (b) are as below: gently pipetting the liquid in the upper layer in the centrifuge tubes with an electric pipette gun equipped with a 10 ml pipette, and gently pipetting the last approximately 0.5 ml liquid in the upper layer with a 1 ml manual pipette, so as to pipette the buffy-coat layer out as much as possible and pipette the erythrocytes in the lower layer as little as possible.

4. The method of separating hematopoietic stem cells from umbilical cord blood according to claim 1, wherein, in the step (c), the centrifugal force for the centrifugation of the upper liquid layer and the lower erythrocyte layer is approximately 747 g, and the time for centrifugation is approximately 10 min.

5. The method of separating hematopoietic stem cells from umbilical cord blood according to claim 1, wherein, in the step (d), the superficial buffy-coat layer is metered with the plasma in the upper layer to approximately 30 mL, the centrifugal force is approximately 747 g, and the time for centrifugation is approximately 10 min.

6. The method of separating hematopoietic stem cells from umbilical cord blood according to claim 2, wherein, in the step (d), the superficial buffy-coat layer is metered with the plasma in the upper layer to approximately 30 mL, the centrifugal force is approximately 747 g, and the time for centrifugation is approximately 10 min.

7. The method of separating hematopoietic stem cells from umbilical cord blood according to claim 3, wherein, in the step (d), the superficial buffy-coat layer is metered with the plasma in the upper layer to approximately 30 mL, the centrifugal force is approximately 747 g, and the time for centrifugation is approximately 10 min.

8. The method of separating hematopoietic stem cells from umbilical cord blood according to claim 4, wherein, in the step (d), the superficial buffy-coat layer is metered with the plasma in the upper layer to approximately 30 mL, the centrifugal force is approximately 747 g, and the time for centrifugation is approximately 10 min.

9. The method of separating hematopoietic stem cells from umbilical cord blood according to claim 1, wherein, in the step (e), the plasma in the upper layer is added for metering to 20 mL.

10. The method of separating hematopoietic stem cells from umbilical cord blood according to claim 2, wherein, in the step (e), the plasma in the upper layer is added for metering to approximately 20 mL.

11. The method of separating hematopoietic stem cells from umbilical cord blood according to claim 3, wherein, in the step (e), the plasma in the upper layer is added for metering to approximately 20 mL.

12. The method of separating hematopoietic stem cells from umbilical cord blood according to claim 4, wherein, in the step (e), the plasma in the upper layer is added for metering to approximately 20 mL.

13. The method of separating hematopoietic stem cells from umbilical cord blood according to claim 1, wherein, the freezing medium used in the step (e) is prepared as below: dextran is mixed with dimethyl sulfoxide (DMSO) at a ratio of approximately 1:1, with dextran firstly added and then DMSO, and then precooled to get the freezing medium.

14. The method of separating hematopoietic stem cells from umbilical cord blood according to claim 2, wherein, the freezing medium used in the step (e) is prepared as below: dextran is mixed with dimethyl sulfoxide (DMSO) at a ratio of approximately 1:1, with dextran firstly added and then DMSO, and then precooled to get the freezing medium.

15. The method of separating hematopoietic stem cells from umbilical cord blood according to claim 3, wherein, the freezing medium used in the step (e) is prepared as below: dextran is mixed with dimethyl sulfoxide (DMSO) at a ratio of 1:1, with dextran firstly added and then DMSO, and then precooled to get the freezing medium.

16. The method of separating hematopoietic stem cells from umbilical cord blood according to claim 4, wherein, the freezing medium used in the step (e) is prepared as below: dextran is mixed with dimethyl sulfoxide (DMSO) at a ratio of approximately 1:1, with dextran firstly added and then DMSO, and then precooled to get the freezing medium.

17. The method of separating hematopoietic stem cells from umbilical cord blood according to claim 1, wherein, in the step (f), the temperature for storage in the liquid nitrogen tank is approximately −196° C.

18. The method of separating hematopoietic stem cells from umbilical cord blood according to claim 2, wherein, in the step (f), the temperature for storage in the liquid nitrogen tank is approximately −196° C.

19. The method of separating hematopoietic stem cells from umbilical cord blood according to claim 3, wherein, in the step (f), the temperature for storage in the liquid nitrogen tank is approximately −196° C.

20. The method of separating hematopoietic stem cells from umbilical cord blood according to claim 4, wherein, in the step (f), the temperature for storage in the liquid nitrogen tank is approximately −196° C.
